(19) United States
(12) Patent Application Publication
(10) Pub. No.: US 2012/0238760 A1
(43) Pub. Date: Sep. 20, 2012

(54) Method for Producing 5,5-Disubstituted 2-Isoxazolines

(75) Inventor: Timo Frassetto, Mannheim (DE)

(73) Assignee: BASF SE, Ludwigshafen (DE)

(21) Appl. No.: 13/511,090

(22) PCT Filed: Nov. 26, 2009
(86) PCT No.: PCT/EP09/65927

§ 371 Date: May 21, 2012

(51) Int. Cl. C07D 261/04

(52) U.S. Cl. 548/240

(57) ABSTRACT

The present invention relates to a process for preparing 5,5-disubstituted 2-isoxazolines of the formula (I)

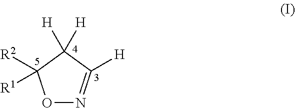

where
R¹, R² are each independently C₁-C₆-alkyl or C₁-C₄-haloalkyl; or R¹ and R² together form a C₂-C₅-alkanediyl chain which may be mono- to tetrasubstituted by C₁-C₄-alkyl and/or interrupted by oxygen or by optionally C₁-C₄-alkyl-substituted nitrogen.

The present invention relates to a process for preparing 5,5-disubstituted 2-isoxazolines of the formula (I)

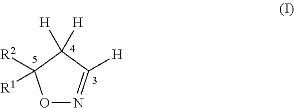

where
R¹, R² are each independently C₁-C₅-alkyl or C₁-C₄-haloalkyl; or R¹ and R² together form a C₂-C₅-alkanediyl chain which may be mono- to tetrasubstituted by C₁-C₄-alkyl and/or interrupted by oxygen or by optionally C₁-C₄-alkyl-substituted nitrogen.

2-Isoxazolines are typically prepared by 1,3-dipolar cycloaddition of nitrile oxides onto alkenes (1) or the reaction of α,β-unsaturated ketones with hydroxylamines (2) (Lang, S. A.; Lin, Y.-I. in Comprehensive Heterocyclic Chemistry, Vol. 6, Katritzky, A. R.; Rees, C. W., Eds.; Pergamon: Oxford, 1984, 88-98). A disadvantage in the case of method (1) has been found to be the tendency of the nitrile oxides to dimerize. Synthesis strategy (2) frequently gives rise to product mixtures.

A further method of preparing 2-isoxazolines is the intramolecular rearrangement of O-propargylhydroxylamines, which, however, requires a complicated synthesis of the starting compound (Synlett 2006 (3), 463).

The cyclization of β-haloketone oximes likewise requires the formation of the starting material by multistage synthesis and has only found isolated uses (J. Org. Chem. 1970 (35), 2065).

Particularly the synthesis of 3-unsubstituted 2-isoxazolines by method (1) is found to be difficult, since the fulminic acid needed for the cycloaddition is stable only at low temperatures and polymerizes readily. The activation of nitromethane with isocyanates to obtain the nitrile oxide does not succeed (Chem. Ber. 106 (1973), 3291), and the activation of nitromethane with trimethylsilyl chloride affords, in the few published examples, only moderate yields (J. Org. Chem. 66 (2001), 2296 and Tetrahedron 39 (1983), 2247).

The direct reaction of α,β-unsaturated aldehydes with hydroxylamines to give 2-isoxazolines by method (2) is unknown. A two-stage alternative consists in the addition of N-hydroxyurea onto α,β-unsaturated aldehydes in aqueous methanolic solution, followed by the acidic elimination of carbamic acid to give 2-isoxazolines (Tetrahedron Lett. 28 (1975), 2337, Bull. Soc. Chim. 5-6 Pt. 2 (1979), 281).

A one-stage reaction which has been published is the reaction of α,β-unsaturated aldehydes with oximes in the presence of an acid or of a combination of acid and of a nitrogen-containing base to give 5-monosubstituted 2-isoxazolines (Synlett 2008 (6), 827), but with a low space-time yield and exclusively for β-monosubstituted aldehydes.

The failure of the addition of oximes onto α-branched, α,β-unsaturated aldehydes suggests the significance of steric factors for this reaction.

The analogous Michael addition of alcohols onto α,β-unsaturated aldehydes with organic catalysis shows a great dependence of the yield on the steric demand of the substrate in the β position, large substituents giving rise to the smallest yields (Tetrahedron Let. 47 (2006) 3039-3041).

The acid-catalyzed addition of alcohols onto α,β-unsaturated ketones also gives rise to satisfactory yields only in the case of terminal or β-monosubstituted enones (Org. Lett., Vol.5, No. 12, 2141-44).

5,5-Disubstituted 2-isoxazolines of the formula (I) are important intermediates for the preparation of active agrochemical and pharmaceutical compounds.

It was accordingly an object of the present invention to provide an inexpensive, economically viable and safe process, suitable for industrial scale use, for preparing 5,5-disubstituted 2-isoxazolines of the formula (I).

It has been found that, surprisingly, 5,5-disubstituted 2-isoxazolines of the formula (I)

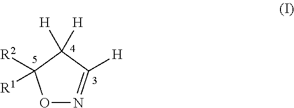

are obtained in a one-stage process in very good yield from β-disubstituted aldehydes, where
R¹, R² are each independently C₁-C₈-alkyl or C₁-C₄-haloalkyl; or R¹ and R² together form a C₂-C₅-alkanediyl chain which may be mono- to tetrasubstituted by C₁-C₄-alkyl and/or interrupted by oxygen or by optionally C₁-C₄-alkyl-substituted nitrogen;
which comprises reacting an oxime of the formula (II)

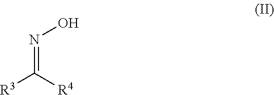

where
R³, R⁴ are each independently hydrogen, C₁-C₆-alkyl, C₁-C₄-alkylcarbonyl, hydroxyimino-C₁-C₄-alkyl, phenyl, phenyl-C₁-C₄-alkyl or phenyl-C₂-C₄-alkenyl, where the phenyl rings may be mono- or polysubstituted by C₁-C₄-alkyl, C₁-C₄-alkoxy, di-C₁-C₄-alkylamino, halogen, hydroxyl or nitro; or R³ and R⁴ together form a C₂-C₅-alkanediyl chain;
with a carbonyl compound of the formula (III)

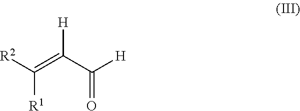

where R¹ and R² are each as defined above;
in the presence of an acid catalyst or an acid-base catalyst and optionally in the presence of an organic solvent.

The present application therefore provides the process according to the invention for preparing 5,5-disubstituted 2-isoxazolines of the formula (I).

5,5-Disubstituted 2-isoxazolines are intermediates in a process for preparing oxazole herbicides of the formula (IV)

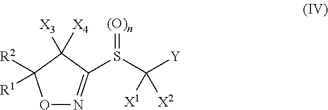

where the variables are each defined as follows:
n is 0, 1 or 2;
X¹, X², X³, X⁴ are each independently hydrogen, fluorine or chlorine; and
Y is phenyl, 6-membered heteroaryl having one to three nitrogen atoms or 5-membered heteroaryl having one to three heteroatoms selected from the group of oxygen, nitrogen and sulfur, where phenyl and heteroaryl may each be substituted by one to five substituents selected from the group of halogen, nitro, cyano, C₃-C₆-cycloalkyl, C₁-C₄-haloalkyl, carboxy-C₁-C₄-alkyl, sulfonyl-C₁-C₄-alkyl, C₂-C₆-alkenyl, C₂-C₆-alkynyl, C₁-C₄-alkoxy, C₁-C₄-haloalkoxy, C₂-C₆-alkenyloxy, C₂-C₆-alkynyloxy and C₁-C₄-alkylcarbonyloxy; and R¹, R² are each independently C₁-C₆-alkyl or C₁-C₄-haloalkyl; or R¹ and R² together form a C₂-C₅-alkanediyl chain which may be mono- to tetrasubstituted by C₁-C₄-alkyl and/or interrupted by oxygen or by optionally C₁-C₄-alkyl-substituted nitrogen.

Oxazole herbicides of the formula (IV) are known from WO 02/062770 and WO 01/012613.

The present application therefore also relates to a process for preparing oxazole herbicides of the formula (IV), comprising the preparation of 5,5-disubstituted 2-isoxazolines of the formula (I) by the process according to the invention.

Further embodiments of the present invention can be inferred from the claims, the description and the examples. It will be appreciated that the features of the inventive subject matter which have been specified above and those which are yet to be explained below are usable not only in the combination specified in the particular case, but also in other combinations, without leaving the scope of the invention.

The organic molecular moieties specified for the substituents constitute collective terms for individual lists of the individual group members. Hydrocarbon chains may be straight or branched. Unless stated otherwise, halogenated substituents preferably bear one to five identical or different halogen atoms.

The definition “halogen” in each case represents fluorine, chlorine, bromine or iodine, preferably fluorine or chlorine.

Examples of further definitions include:

Alkyl and the alkyl moieties of carboxyalkyl, sulfonylalkyl, phenylalkyl, arylalkyl, heterocyclylalkyl, heteroarylalkyl, alkoxy, alkylcarbonyl, hydroxyiminoalkyl, alkylamino, alkylcarbonyloxy, alkylsilyl and alkylsilyloxy are each a saturated, straight-chain or branched hydrocarbon group having 1 to 6 or 1 to 4 carbon atoms, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dinnethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl and isomers thereof. C₁-C₄-Alkyl comprises, for example, methyl, ethyl, propyl, 1-methylethyl, butyl, 1-methylpropyl, 2-methylpropyl or 1,1-dimethylethyl.

Cycloalkyl denotes monocyclic saturated hydrocarbon groups having three or more carbon atoms, for example 3 to 6 carbon ring members, such as cyclopropyl, cyclobutyl, cyclopentyl and cyclohexyl.

Haloalkyl and the haloalkyl moieties of haloalkoxy each represent partly or fully halogenated alkyl, where the halogen atom(s) is/are especially fluorine, chlorine and/or bromine, for example chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2-chloro-2-fluoroethyl, 2,2,2-trifluoroethyl, 2-chloro-1,1,2-trifluoroethyl, 2-chloro-2,2-difluoroethyl, 2-bromo-2,2-difluoroethyl, 2,2-dichloro-2-fluoroethyl, 2,2,2-trichloroethyl, 1,1,2,2-tetrafluoroethyl, 1,1,2,2-tetrachloroethyl, pentafluoroethyl, 2,2,3,3-tetrafluoro-1-propyl, 1,1,2,3,3,3-hexafluoro-1-propyl, 1,1,1,3,3,3-hexafluoro-2-propyl, heptafluoro-1-propyl, heptafluoro-2-propyl, 2,2,3,3,4,4,4-heptafluoro-1-butyl or nonafluoro-1-butyl.

Alkenyl and the alkenyl moieties of phenylalkenyl and alkenyloxy are each a monounsaturated, straight-chain or branched hydrocarbon group having two to six or two to four carbon atoms and a double bond in any position, for example ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl.

Alkynyl and the alkynyl moieties of alkynyloxy each denote straight-chain or branched hydrocarbon groups having two or more carbon atoms, for example 2 to 4, 2 to 6, or 3 to 6 carbon atoms, and one or two triple bonds in any position, but not adjacent positions, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl.

Aryl denotes a mono- to tricyclic aromatic carbocycle having 6 to 14 ring members, for example phenyl, naphthyl and anthracenyl.

Heteroaryl denotes a 5- or 6-membered aromatic ring system having one to four nitrogen atoms or having one to three nitrogen atoms and one oxygen or sulfur atom, or having one oxygen or sulfur atom.

Heterocyclyl denotes a saturated, partially unsaturated or aromatic heterocyclic ring having three or more carbon atoms, e.g. 3-, 4-, 5- or 6-membered heterocyclic ring which comprises one to four identical or different heteroatoms selected from the group of oxygen, sulfur and nitrogen, and may be bonded via C or N; where one sulfur in the heterocyclyl may be oxidized to S═O or S(═O)2, and where a bicyclic ring system may be formed with a fused-on phenyl ring or with a C₃-C₆-carbocycle or with a further 5- to 6-membered heterocycle.

In the process according to the invention, preference is given to using compounds of the formulae (II) and (III), where the variables, in each case alone or in combination, are each defined as follows:

R¹ is C₁-C₆-alkyl or C₁-C₄-haloalkyl;
R² is C₁-C₆-alkyl or C₁-C₄-haloalkyl;
R³ is hydrogen, C₁-C₆-alkyl, C₁-C₄-alkylcarbonyl, hydroxyimino-C₁-C₄-alkyl; and
R⁴ is C₁-C₆-alkyl; or R³ and R⁴ form a C₂-C₅-alkanediyl chain.

Particular preference is given to using compounds of the formulae (II) and (III), where the variables, in each case alone or in combination, are each defined as follows:

R¹ is C₁-C₄-alkyl, especially methyl or ethyl, more preferably methyl;
R² is C₁-C₄-alkyl, especially methyl or ethyl, more preferably methyl;
R³ is hydrogen, C₁-C₄-alkyl, especially methyl, ethyl, isopropyl or isobutyl, more preferably methyl and ethyl; and

R⁴ is C₁-C₄-alkyl, especially methyl or ethyl; or R³ and R⁴ form a C₂-C₅-alkanediyl chain.

Exceptional preference is given to using compounds of the formulae (II) and (III), where the variables, in each case alone or in combination, are each defined as follows:

R¹ is methyl;
R² is methyl;
R³ is methyl or ethyl;
R⁴ is methyl or ethyl.

A particularly preferred 5,5-disubstituted 2-isoxazoline of the formula (I) which is prepared by the process according to the invention is 5,5-dimethyl-2-isoxazoline (Ia), where R¹ and R² in formula (I) are each methyl.

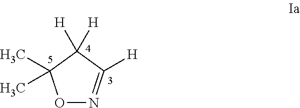

The 5,5-disubstituted 2-isoxazoline of the formula (Ia) where R¹ and R² are each defined as methyl is preferably used as an intermediate in processes for preparing oxazole herbicides of the formula (IVB) where

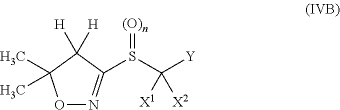

n is 1 or 2;
X¹, X²are each independently hydrogen or fluorine; and
Y is phenyl, where phenyl may be substituted by one to three substituents selected from the group of halogen, C₁-C₄-alkyl, C₁-C₄-haloalkyl and C₁-C₄-alkoxy.

More particularly, the 5,5-disubstituted 2-isoxazoline of the formula (la) where R¹ and R² are each defined as methyl is also used as an intermediate in processes for preparing oxazole herbicides of the formula (IVA) where

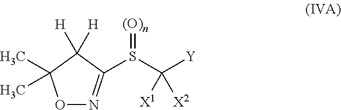

n is 1 or 2;
X¹, X²are each independently hydrogen or fluorine; and
Y is pyrazolyl which may be substituted by one to three substituents selected from the group of C₁-C₄-alkyl, C₁-C₄-haloalkyl and C₁-C₄-haloalkoxy.

Exceptionally preferably, the 5,5-disubstituted 2-isoxazoline of the formula (Ia) is used as an intermediate in processes for preparing the oxazole herbicides of the formulae (IVa) and (IVb).

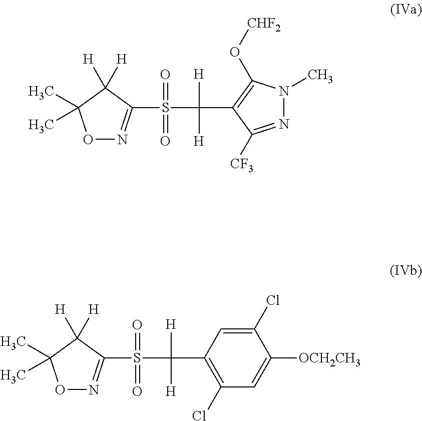

Proceeding from intermediates of the formula (I), the further steps in the process for preparing oxazole herbicides of the formula (IV) are already known per se to those skilled in the art or have been described in the literature.

For the compound of the formula (IVa), one possible synthesis can be illustrated as follows:

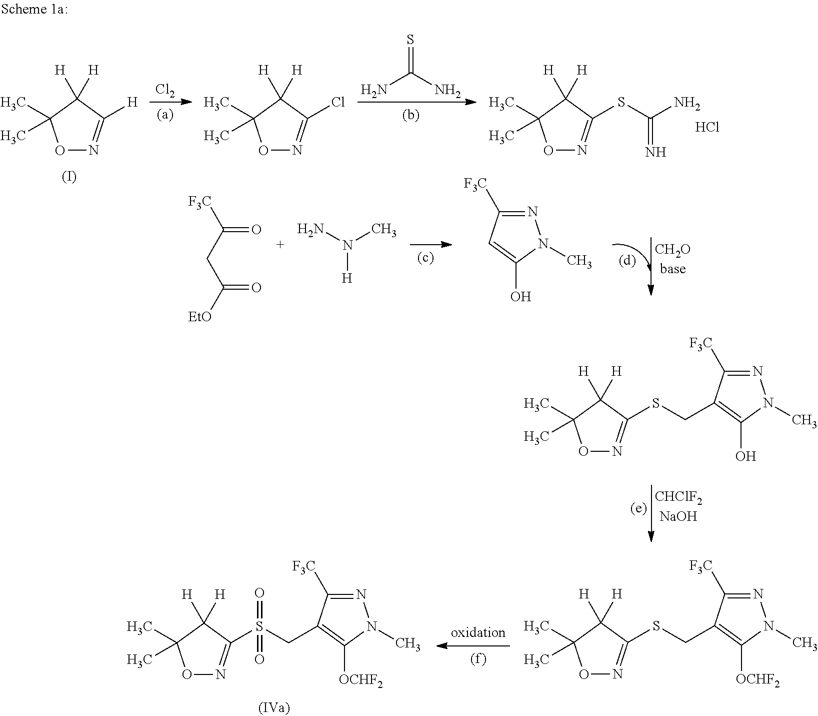

The chlorination (a) of 3-unsubstituted isoxazolines with chlorine gas is known to those skilled in the art (J. Org. Chem. 53 (1988), 4074-81). The subsequent reaction with thiourea to give thiocarboxamidine salts (b) is described in EP 1 829 868. The preparation of the pyrazole (c) from dicarbonyl compounds and hydrazines is demonstrated, for example, in JP2007/031342. The reaction of the hydroxyl-substituted pyrazole with formaldehyde, followed by the reaction with thiocarboxamidine salts (d) is described in CA 2 560 936 (WO2005/095352). The alkylation of the hydroxyl group (e) is known to those skilled in the art from JP2007/246396. The final oxidation of the sulfur to the sulfone (f) is employed, for example, in EP 1 405 853.

For the compound of the formula (IVb), one possible synthesis can be illustrated as follows:

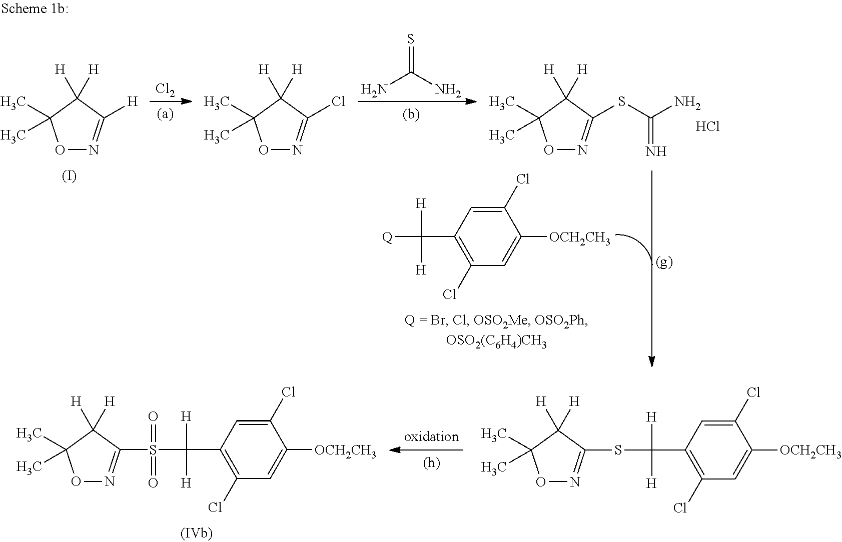

The chlorination (a) of 3-unsubstituted isoxazolines with chlorine gas is known to the person skilled in the art (J. Org. Chem. 53 (1988), 4074-81). The subsequent reaction with thiourea to give thiocarboxamidine salts (b) is described in EP 1 829 868. The nucleophilic substitution of benzylic bromides (g) was demonstrated, for example, in

WO2007/096576. The final oxidation of the sulfur to give the sulfone (h) is applied, for example, in EP 1 405 853.

In the process according to the invention, the oxime of the formula (II) is reacted with the carbonyl compound of the formula (III) in the presence of an acid or an acid-base catalyst and optionally in the presence of an organic solvent (scheme 2):

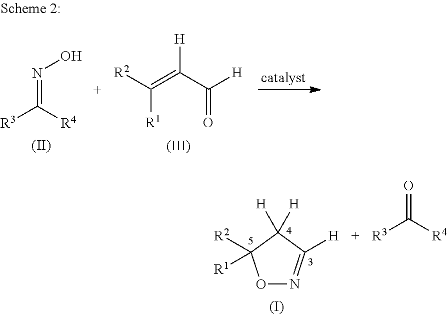

The oximes of the formula (II) are either commercially available or can be prepared, for example, according to Yamane, M.; Narasaka, K., in Science of Synthesis, 27 (2004), p. 605. The carbonyl compounds of the formula (III) are likewise commercially available or can be prepared, for example, according to Escher, I.; Glorius, F., in Science of Synthesis, 25 (2006), p. 733.

In general, the process according to the invention for preparing the compound of the formula (I) is performed under the following conditions:

The oxime of the formula (II) and the carbonyl compound of the formula (III) are used in the process according to the invention in a molar ratio of 3:1 to 1:3. The excess of one of the two components is preferably up to 20 mol %, especially of the carbonyl compound of the formula (III). The preferred molar ratio of the oxime (II) to the carbonyl compound (III) is correspondingly 1.0:0.8 to 1.0:1.2, more preferably about 1.0:1.0 to 1.0:1.1.

The reaction of the oxime of the formula (II) and of the carbonyl compound of the formula (III) takes place in the presence of a catalyst. Suitable catalysts are, particular acids (acid catalyst) or mixtures of particular acids and particular bases (acid-base catalyst).

The acid-catalyzed process according to the invention can be illustrated diagrammatically as follows:

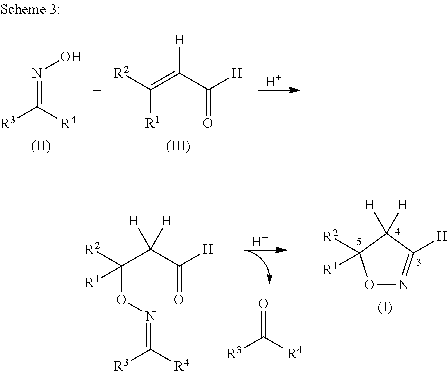

Suitable acid catalysts are proton donors (Brønsted acids), for example inorganic and organic acids. Examples of inorganic acids are hydrohalic acids and oxygen acids, especially hydrochloric acid, sulfuric acid, nitric acid, phosphoric acid, phosphonic acid and phosphinic acid.

Examples of organic acids are aliphatic and aromatic acids such as alkylsulfonic acids, arylsulfonic acids, mono-C₁-C₆-alkyl phosphates, di-C₁-C₅-alkyl phosphates, monoaryl phosphates, diaryl phosphates, alkylcarboxylic acids, haloalkylcarboxylic acids and heterocyclylcarboxylic acids, especially methanesulfonic acid, p-toluenesulfonic acid, citric acid, trifluoroacetic acid and proline.

In general, the reaction proceeds under acid catalysis with a good yield with those acids whose pKa is less than 3.5.

When the process according to the invention is performed only with acid catalysis, preference is given to using strong acids such as phosphoric acid, mono-C₁-C₆-alkyl phosphates, di-C₁-C₆-alkyl phosphates, monoaryl phosphates, diaryl phosphates, sulfuric acid, sulfonic acids or trifluoroacetic acid.

The inventive acid-base-catalyzed process could proceed according to the following scheme:

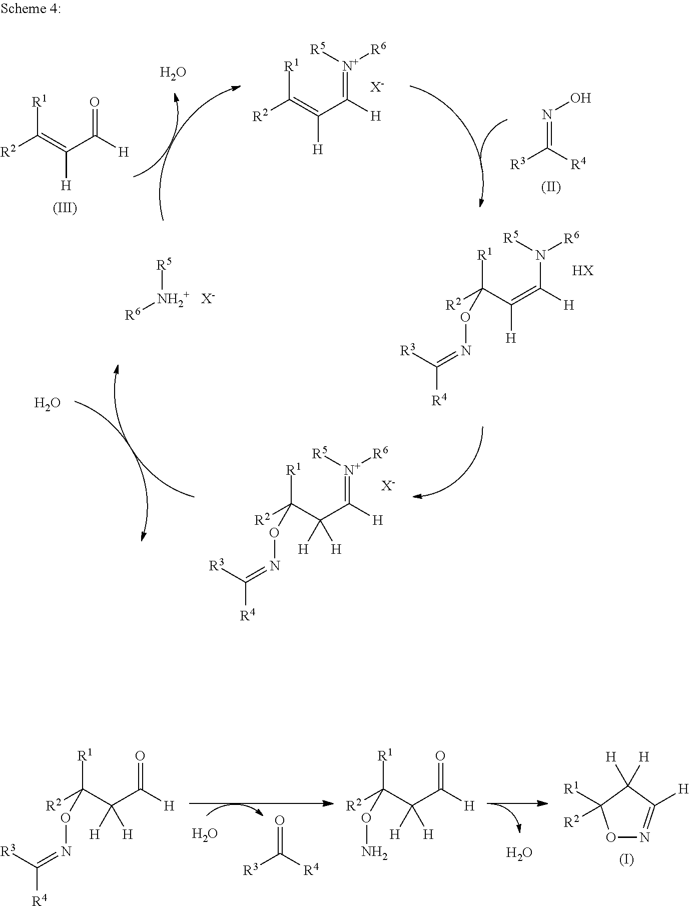

Suitable acid-base catalysts are mixtures of the above-described acids and particular bases, acid and base being usable separately from one another or as an acid addition salt.

Suitable bases have been found to be compounds which comprise one or more heteroatoms, for example nitrogen, oxygen, sulfur or phosphorus, nitrogen being a preferred heteroatom.

Examples of such bases are primary or secondary amines of the formula (V)

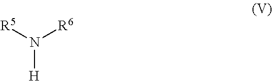

where R⁵ and R⁶ are each independently C₁-C₆-alkyl, C₃-C₆-cycloalkyl, C₂-C₆-alkenyl, C₂-C₆-alkynyl, tri-C₁-C₆-alkylsilyl, aryl, aryl-C₁-C₆-alkyl, heteroaryl, heteroaryl-C₁-C₆-alkyl, and where the aryl and heteroaryl moieties of the substituents may themselves be substituted by one to three substituents selected from the group of halogen, nitro, cyano, C₁-C₄-alkyl, C₃-C₆-cycloalkyl, C₁-C₄-haloalkyl, carboxy-C₁-C₄-alkyl, C₂-C₆-alkenyl, C₂-C₆-alkynyl, C₁-C₄-alkoxy, C₁-C₄-haloalkoxy, C₂-C₆-alkenyloxy, C₂-C₆-alkynyloxy, C₁-C₄-alkylcarbonyloxy;
and where R⁵ may additionally be hydrogen.

Preference is given to compounds of the formula (V) in which R⁵ and R⁶ are each independently C₁-C₆-alkyl, C₃-C₆-cycloalkyl, tri-C₁-C₆-alkylsilyl, aryl or aryl-C₁-C₆-alkyl; and where the aryl moieties of the substituents may themselves be substituted by one to three substituents selected from the group of halogen, nitro, cyano, C₁-C₄-alkyl, C₃-C₆-cycloalkyl, C₁-C₄-haloalkyl, carboxy-C₁-C₄-alkyl, C₂-C₆-alkenyl, C₂-C₆-alkynyl, C₁-C₄-alkoxy, C₁-C₄-haloalkoxy, C₂-C₆-alkenyloxy, C₂-C₆-alkynyloxy, C₁-C₄-alkylcarbonyloxy; and where R⁵ may additionally be hydrogen.

Particular preference is given to compounds of the formula (V) in which R⁵ and R⁶ are each independently methyl, ethyl, propyl, 1-methylethyl, butyl, cyclopentyl, cyclohexyl, phenyl, naphthyl, benzyl or trimethylsilyl; and where R⁵ may additionally be hydrogen; for example N-methylaniline.

Alternatively, R⁵ and R⁶ together may form a ring structure of the formula (VI)

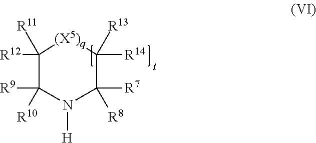

where

### X⁵ is O, S, NR¹⁵ or CR¹⁶R¹⁷;

q is 0 or 1;
t is 0 or 1; and
R⁷, R⁸, R⁹, R¹⁰, R¹¹, R¹², R¹³, R¹⁴, R¹⁶, R¹⁷ are each independently selected from hydrogen, hydroxyl, carboxyl, amino, nitro, aminocarbonyl, C₁-C₆-alkyl, C₁-C₆-alkoxy, C₁-C₆-alkylcarbonyl, mono-C₁-C₆-alkylamino, di-C₁-C₆-alkylamino, aryl, heteroaryl, aryl-C₁-C₆-alkoxy, and where C₁-C₆-alkyl may itself be substituted by aryl, heteroaryl, heterocyclyl or trimethylsilyloxy, and where the aryl, heterocyclyl and heteroaryl moieties of the substituents may themselves be substituted by one to three substituents selected from the group of halogen, nitro, cyano, C₁-C₄-alkyl, C₃-C₆-cycloalkyl, C₁-C₄-haloalkyl, carboxy-C₁-C₄-alkyl, C₂-C₆-alkenyl, C₂-C₆-alkynyl, C₁-C₄-alkoxy, C₁-C₄-haloalkoxy, C₂-C₆-alkenyloxy, C₂-C₆-alkynyloxy and C₁-C₄-alkylcarbonyloxy;
or R¹¹ and R¹² and/or R¹³ and R¹⁴ form, together with the carbon atom to which they are bonded, a keto group; and R¹⁵ is selected from hydrogen, C₁-C₆-alkyl, aryl-C₁-C₆-alkyl.

Preference is given to amines of the formula (V) or (VI), where R⁵ and R⁶ together with the NH group form an imidazolin-5-one ring of the formula (VII)

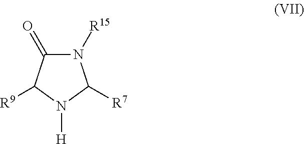

where the substituents are each as defined above.

Particular preference is given here to compounds in which R⁷ and R⁹ are each independently selected from C₁-C₆-alkyl and aryl-C₁-C₆-alkyl, preferably from methyl, ethyl, 1-methylethyl, 1,1-dimethylethyl and phenylmethyl.

In general, the catalysts are added to the reaction mixture in a catalytic amount. In one embodiment of the invention, the molar ratio of the compound of the formula II to the acid catalysts or acid-base catalysts is less than 1:0.1. In a preferred embodiment, the molar ratio is less than 1:0.05, in a particularly preferred embodiment less than 1:0.02.

The oxime of the formula (II) and the carbonyl compound of the formula (III) can, in accordance with the invention, be reacted either without addition of a solvent or with addition of a suitable solvent.

In one embodiment of the process according to the invention, the oxime of the formula (II) and the carbonyl compound of the formula (III) are reacted with one another with addition of a solvent.

Suitable solvents are organic solvents, for example, aromatic hydrocarbons such as toluene, o-, m-, p-dimethylbenzene, 1,3,5-trimethylbenzene, ethylbenzene, chlorobenzene, o-,m-,p-dichlorobenzene, halogenated aliphatic hydrocarbons such as tetrachloroethane, trichloromethane, dichloromethane and dichloroethene, aliphatic hydrocarbons such as pentane, hexane, heptane, octane, cyclopentane, methylcyclopentane and cyclohexane, ethers such as diethyl ether, methyl tert-butyl ether, tetrahydrofuran and dioxane, alcohols such as methanol and ethanol, esters such as ethyl acetate, nitriles such as acetonitrile, or mixtures of the solvents mentioned.

Preferred solvents are aromatic hydrocarbons, halogenated aliphatic hydrocarbons, aliphatic hydrocarbons, ethers and alcohols.

Particularly preferred solvents are aromatic hydrocarbons, especially toluene, chlorobenzene, dichlorobenzene and mixtures of these solvents.

In a preferred embodiment of the process according to the invention, the proportion of the solvent in the reaction mixture, i.e. before the start of the reaction, is less than 80% by weight. In a particularly preferred embodiment the proportion of the solvent is less than 50% by weight, most preferably less than 10% by weight.

In a further, particularly preferred embodiment of the process according to the invention, the oxime of the formula (II) and the carbonyl compound of the formula (III) are reacted with one another without addition of a solvent.

In general, the sequence in which the oxime of the formula (II), the carbonyl compound of the formula (III), the catalyst and optionally the solvent are initially charged in or added to the reaction vessel is unimportant. In one embodiment of the invention, the oxime of the formula (II) and the carbonyl compound of the formula (III) and, optionally the solvent are initially charged and the desired temperature is established. Then the catalyst is added.

In another embodiment of the invention, the oxime of the formula (II), the catalyst and optionally the solvent are initially charged and the desired temperature is established. Then the carbonyl compound of the formula (III) is added.

Addition is understood to mean the addition of a substance, either a little at a time or continuously. The catalyst and the carbonyl compound of the formula (III) are added preferably without solvent or dissolved in an organic solvent as defined above in the course of the reaction.

It is normal to work at a reaction temperature of −40 to 100° C., preferably of −20 to 60° C., especially of 0 to 30° C.

The reaction mixture can be supplied directly to other processes without further workup. The reaction product, the 5,5-disubstituted 2-isoxazoline of the formula (I), can also be removed from the reaction mixture, for example by direct distillation, extraction or chromatography, preferably by distillation.

### EXAMPLES

### Example 1

### (Acid-Base Catalysis)

Synthesis of 5,5-dimethyl-2-isoxazoline

10.0 g (0.137 mol; 100 mol %) of acetone oxime were admixed with 12.7 g (0.151 mol, 110 mol %) of 3-methyl-2-butenal and cooled to 10° C. Over the course of 3 h, a mixture of 0.13 g (1.2 mmol, 0.9 mol %) of N-methylaniline and 0.14 g (1.2 mmol, 0.9 mol %) of trifluoroacetic acid was added a little at a time to this mixture, and the temperature was increased to 22° C. after 20% of the addition. After 3 h, the product of value was isolated from the reaction mixture by fractional distillation under reduced pressure. Boiling point 44-48° C. at 17-18 mbar. 10.0 g of 5,5-dimethyl-2-isoxazoline were obtained, 89% pure according to ¹H NMR (66%).

¹H NMR (CDCI3): 1.40 (s, 6H), 2.75 (d, 2H), 7.06 (br s, 1 H).

### Example 2

### (Acid-Base Catalysis)

Synthesis of 5,5-dimethyl-2-isoxazoline

50.0 g (0.684 mol, 100 mol %) of acetone oxime were admixed with 62.6 g (0.744 mol, 109 mol %) of 3-methyl-2-butenal and cooled to 10-15° C. Over the course of 48 h, 1.5 g (6.8 mmol, 1 mol %) of N-methylanilinium trifluoroacetate were added to this mixture in 0.1 g portions. The product of value was subsequently isolated from the reaction mixture by fractional distillation under reduced pressure. Boiling point 44-48° C. at 17-18 mbar. 56.9 g of 5,5-dimethyl-2-isoxazoline were obtained, >91% pure according to ¹H NMR (76%).

¹H NMR (CDCl₃): 1.40 (s, 6H), 2.75 (d, 2H), 7.06 (br s, 1 H).

### Example 3

### (Acid Catalysis)

Synthesis of 5,5-dimethyl-2-isoxazoline

4.35 g of acetone oxime (59.5 mmol, 100 mol %) and 5.27 g of 3-methyl-2-butenal (62.6 mmol, 105 mol %) were mixed and admixed with 0.13 g of trifluoroacetic acid (1.1 mmol, 1.9 mol %). The mixture was stirred at room temperature for 60 h and the product was distilled under reduced pressure (46° C., 18 mbar). 4.7 g of colorless oil were obtained, with a purity of >95% according to NMR (45.0 mmol, 76%).

### Example 4

### (Acid-Base Catalysis)

Synthesis of 5,5-dimethyl-2-isoxazoline

0.4 g of (2S,5S)-2-tert-butyl-3-methyl-5-benzyl-4-imidazolinone (1.6 mmol, 1 mol %) was initially charged in 50 ml of n-pentane and admixed at 0° C. with 0.19 g of trifluoroacetic acid (1.6 mmol, 1 mol %). The mixture was stirred at -3 - 0° C. for 30 minutes. 11.9 g of acetone oxime (0.163 mol, 100 mol %) were added at 0° C. to the resulting suspension, the mixture was warmed to 20° C., and 16.5 g of 3-methyl-2-butenal (0.196 mol, 120 mol %) were added dropwise within 5 min. The mixture was stirred at this temperature for 16 h, and the product was isolated by distillation. 15.5 g of 5,5-dimethyl-2-isoxazoline were obtained with a purity (GC) of 88%, corresponding to a yield of 84%.

1-10. (canceled)

11. A process for preparing 5,5-disubstituted 2-isoxazolines of the formula (I) 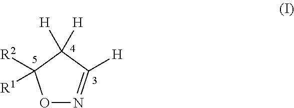
wherein
R¹, R² are each independently C₁-C₆-alkyl or C₁-C₄-haloalkyl; or
R¹ and R² together form a C₂-C₅-alkanediyl chain which may be mono- to tetrasubstituted by C₁-C₄-alkyl and/or interrupted by oxygen or by optionally C₁-C₄-alkyl-substituted nitrogen;
which comprises reacting an oxime of the formula (II) 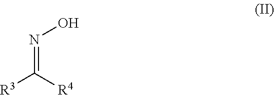
where
R³, R⁴ are each independently hydrogen, C₁-C₆-alkyl, C₁-C₄-alkylcarbonyl, hydroxyimino-C₁-C₄-alkyl, phenyl, phenyl-C₁-C₄-alkyl or phenyl-C₂-C₄-alkenyl, where the phenyl rings may be mono- or polysubstituted by C₁-C₄-alkyl, C₁-C₄-alkoxy, di-C₁-C₄-alkylamino, halogen, hydroxyl or nitro; or R³ and R⁴ together form a C₂-C₅-alkanediyl chain;
with a carbonyl compound of the formula (III) 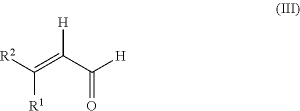
in the presence of an acid catalyst or an acid-base catalyst and optionally in the presence of an organic solvent.

12. The process according to claim 11, wherein
R¹ is C₁-C₆-alkyl or C₁-C₄-haloalkyl;
R² is C₁-C₆-alkyl or C₁-C₄-haloalkyl;
R³ is hydrogen, C₁-C₆-alkyl, C₁-C₄-alkyl carbonyl, hydroxyimino-C₁-C₄-alkyl; and
R⁴ is Cᵢ-C₆-alkyl; or R³ and R⁴ form a C₂-C₅-alkanediyl chain.

13. The process according to claim 11, wherein R¹ and R² are each methyl.

14. The process according to claim 11, wherein R³ and R⁴ are each independently methyl or ethyl.

15. The process according to claim 11, wherein the reaction of the compounds of the formulae (II) and (III) is started with less than 80% by weight of organic solvent in the reaction mixture.

16. The process according to claim 11, wherein the reaction of the compounds of the formulae (II) and (III) is started without addition of an organic solvent in the reaction mixture.

17. The process according to claim 11, wherein the catalyst used is an acid catalyst.

18. The process according to claim 11, wherein the catalyst used is an acid-base catalyst.

19. The process according to claim 11, wherein the molar ratio of the compound of the formula (II) to the catalyst is less than 1:0.1.

20. The process according to claim 11, wherein the molar ratio of the compound of the formula (II) to the catalyst is less than 1:0.02.
